(19) United States
(12) Patent Application Publication
(10) Pub. No.: US 2011/0229961 A1
(43) Pub. Date: Sep. 22, 2011

(54) ACTIVE MICROFLUIDIC SYSTEM FOR IN VITRO CULTURE

(75) Inventors: Len Higashi, Pearl City, HI (US); Dexter Poon, Honolulu, HI (US); Daniel Ling, Honolulu, HI (US); Cathy Owen, Honolulu, HI (US)

(73) Assignee: Nanopoint, Inc., Honolulu, HI (US)

(21) Appl. No.: 13/126,389

(22) PCT Filed: Nov. 4, 2009
(86) PCT No.: PCT/US09/63220

§ 371 Date: May 19, 2011

Related U.S. Application Data
(60) Provisional application No. 61/111,584, filed on Nov. 5, 2008.

(51) Int. Cl. C12M 3/00

(52) U.S. Cl. 435/287.1; 435/289.1

(57) ABSTRACT

Described herein are systems and methods for microfluidic cell support, including a microfluidic cell support system and methods for its use. The microfluidic cell support system can include a base assembly, a manifold assembly, and a microfluidic celltray including a microcirculatory path in fluid communication with the manifold assembly. The microfluidic celltray can be microfluidically closed and mechanically open. In some aspects the microfluidic celltray contains one or more cell wells containing fluid for supporting living cell(s). In some aspects, the microcirculatory path provides active fluid flow between a microfluidic inflow channel and a microfluidic outflow channel.

### BACKGROUND OF THE INVENTION

Controlled environments for growing and maintaining cultured cells are important for a number of in vitro processes, including for assisted reproductive technologies such as in vitro fertilization (IVF). Cells that can be cultured include, for example, embryos that may be used for IVF. Cells being cultured may be studied in themselves or may be subjected to specific interventions whose consequences may then be evaluated. The morphology of the cultured cells may be studied visually, and other methods can be used to assess the cell's physiological condition, such as analysis of its fluid waste. It is desirable to maintain cultured cells under conditions that replicate the in vivo environment, and under conditions that allow beneficial interventions to be made.

Assisted reproductive technologies facilities and other embryo research laboratories recognize the usefulness of systems and methods that mimic the in vivo environment. Currently, the oocytes/embryos are cultured under static conditions that require extensive washings and nutrient media exchanges that can total up to 20 manipulations. These procedural steps have the potential to introduce changes to the microenvironment of the oocyte/embryo such as temperature, pH, osmolality, chemical concentrations, and mechanical forces. Reducing these stresses through the development of technologies that will allow manipulation of the oocyte/embryo under more in vivo like conditions has been a focus for research in recent years.

Currently, embryos for IVF are typically grown in devices like Petri dishes. Petri dishes for embryo culture only allow the manual exchange of fluid by a researcher who typically must remove the dish from an incubator to do this. The embryo experiences sudden changes in its environment when waste material is removed or new culture media is added all at once. Researchers, aware of the low success rate of embryo transfer for IVF, are investigating whether improving the nutrient environment for embryo growth might improve the likelihood that the embryos can implant and grow in vivo.

To mimic the in vivo conditions that an embryo would experience, fluid flow can be used in the in vitro environment. Microfluidic platforms may improve the quality of embryos for IVF by providing environments that simulate the in vivo setting. For example, embryos may be cultured in micro channels, or may be exposed to fluid flow in a microfluidic device. An embryo experiencing a consistent flow of fluid past it in a controlled environment may have a higher chance of successful growth.

Examples of microfluidic platforms useful for in vitro culture include those described in U.S. Pat. Nos. 6,193,647, 6,523,559, and 6,695,765. There remains a need in the art, however, for alternative approaches to microfluidics that may permit the economical and efficient growth and maintenance of embryos. There remains also a need for culture conditions that provide cultured cells with an in-vivo-like environment.

### BRIEF SUMMARY OF THE INVENTION

Disclosed herein are embodiments of a microfluidic cell support system comprising a base assembly, a manifold assembly, and a microfluidic celltray including a microcirculatory path in fluid communication with the manifold assembly, wherein the microfluidic celltray is microfluidically closed and mechanically open. In embodiments, the microfluidic celltray includes one or more cell wells containing fluid for nourishing a living cell, such as an embryo, the fluid having a surface in contact with a gaseous environment, such as an ambient environment. The fluid can be a nutrient fluid for culturing the living cell. In embodiments, the manifold assembly can comprise a hinged cover glass which, when closed, encloses the ambient environment. In embodiments, the ambient environment is a specialized environment with a controlled variable selected from the group consisting of gas composition, humidity, temperature and pressure. In embodiments, the microcirculatory path comprises a microfluidic inflow channel and a microfluidic outflow channel with active fluid flow therebetween. The system may further comprise a sensor circuit to produce data regarding a system parameter, for example a chemical parameter, a temperature parameter, a humidity parameter, and a fluid flow parameter.

Further disclosed herein are methods for supporting a living cell, comprising providing the microfluidic cell support system as set forth above, with the cell support system having a microfluidic celltray comprising a cell well, providing a nutrient fluid selected for supporting a living cell, and perfusing the cell well with the nutrient fluid and introducing the living cell into the cell well containing the nutrient fluid, with the step of perfusing the cell well including establishing active fluid flow from a microfluidic inflow channel to a microfluidic outflow channel across the cell well. In embodiments, the living cell can be an embryo. In embodiments, the cell well is microfluidically closed and mechanically open. In embodiments, the nutrient fluid in the cell well is in contact with an ambient environment. In embodiments, the methods further include a step of regulating a system parameter for the microfluidic cell support system to optimize cell support.

### BRIEF DESCRIPTION OF SEVERAL VIEWS OF THE DRAWINGS

FIG. 1 depicts a perspective view of an embodiment of a microfluidic celltray for in vitro support.

FIG. 2A depicts a perspective view of an embodiment of a microfluidic celltray for in vitro cell culture support, and FIG. 2B diagrams a cross-section of a region of FIG. 2A.

FIG. 3 depicts a block diagram of a system incorporating an exemplary embodiment of a microfluidic celltray for cell culture support.

FIGS. 4A, 4B and 4C depict perspective views of a celltray housing assembly.

### DETAILED DESCRIPTION OF THE INVENTION

Disclosed herein are embodiments of a microfludic celltray for in vitro cell culture support, providing active support to living cells by a system of microfluidic channels that provide active flow to a system of wells wherein the cells reside. In embodiments, the cells can be fertilized embryos, as used, for example, in vitro fertilization (IVF). While the present disclosure addresses certain needs in the art of human IVF, it is understood that the systems and methods disclosed herein can be applied to living cells of any species, including embryos of non-human animals, and including non-embryo living cells for which active support may be desirable.

As used herein, the term “active flow” pertains to a support system for living cells where fluid is delivered to or extracted from some or all of the wells by use of an external positive or negative pressure source, for example a pressure pump, a pressurized tank, a vacuum pump, or the like. As used herein, the term “active support” relates to a support system for living cells where some or all of the wells receive active microfluidic flow. An active microfluidic system may be contrasted to a system using passive microfluidic flow, wherein fluid circulation is impelled by naturally occurring mechanisms such as gravity, capillary action, surface tension or the like to drive the flow. In embodiments, the systems and methods disclosed herein include a microfluidic celltray for embryo support. The microfluidic celltray provides active flow to a plurality of cell wells through a system of microfluidic channels, thereby providing a suitable fluid environment to one or more living cells.

In embodiments, the living cells can be embryos being supported or cultured, for example, for IVF. It is known in the art that supporting and/or culturing living cells, e.g., embryos for IVF, may require changes in the nutrient environment and other environmental conditions. The disclosed systems and methods permit changes in the fluid support for the living cells, including varying concentrations of nutrients, growth factors, and other agents that facilitate growth, and including removing waste materials or other growth inhibitors that may impair growth. Moreover, the disclosed systems and methods permit assessing the environment for cell growth or adjusting this environment. Further disclosed herein is an environment for cell growth where humidity and temperature can be controlled, while permitting ready access to the cells being supported. The microfluidic cell support system described herein includes a block bearing a plurality of wells connected to microfluidic channels. The microfluidic cell support system may also include one or more manifolds in fluid communication with the microfluidic channels. The microfluidic cell support system may also include a fluid delivery system in fluid communication with the manifold and/or the microfluidic channels.

In embodiments, one or more wells in the block contain one or more living cells. In embodiments, the cells can be embryos, human or non-human. The living cells may be surrounded by or otherwise immersed in or in contact with nutrient fluids, as would be understood by those of ordinary skill in the art. The living cells may be suspended in the nutrient fluid, having no other attachment to the surface of the wells. In embodiments, the wells are dimensionally configured so that the living cells, e.g., embryo(s), can float freely in the nutrient fluids, having sporadic contact or no contact with the surfaces of the wells.

In embodiments, one or more microfluidic channels bring nutrient fluids to the wells, and carry spent fluids away from the wells. The block is closed on its inferior aspect by its attachment to a baseplate whose length and width correspond to the dimensions of the block. In embodiments, the baseplate and block may be integrally formed from a single material. In embodiments, the microfluidic channels are positioned within the block, or are positioned on the inferior aspect of the block so that the attachment of the block to the baseplate closes off the channels, or the microfluidic channels may be placed within the baseplate but exist in fluid communication with the block. In embodiments, the baseplate bears indentations or recesses that align with the wells to provide a larger reservoir to contain the embryos. The microfluidic channels contained in the block thus pass fluid over the superior aspect of the recesses in the baseplate, which recesses may contain one or more living cells that have settled therein by gravity. In embodiments, the living cells are suspended in fluid without contacting the walls of the cell well or the recesses. The motion of the fluid flowing through the cell wells can impart motion to the cells residing therein, so that their position and/or orientation changes as they encounter the motion of the fluid in the fluid path, while being confined to the boundaries of the indentation.

The block is open on its superior aspect, so that the wells are open as well. Hence, the microfluidic cell support system is microfluidically closed but mechanically open. This configuration permits access to the living cells, so that they can be readily inserted or removed into the system, while protecting the microfluidic channels and the fluids that they contain from the environment. In embodiments, the fluid in the cell wells is in contact with the ambient environment through their openings on the superior aspect of the block. The term “ambient environment” refers to the environment surrounding the superior aspect of the block, for example, the open atmosphere or a gas-controlled atmosphere if the region surrounding the superior aspect of the block is closed off from the atmosphere and is infused with a selected gas or blend of gases, forming thereby a specialized ambient environment. In embodiments, the ambient environment contacting the cell well fluid can be controlled in its chemical condition, its humidity, its temperature, its pressure, and the like, so as to enhance the growth of cells in the cell wells. After the designated number of living cells has been introduced into one or more cell wells, the cell wells and/or the block may optionally be sealed, for example, with a tape, membrane, or drop of oil.

In embodiments, the block contains a plurality of wells fed and drained by a plurality of microfluidic channels. The plurality of microfluidic channels provides an active flow to at least a portion of the plurality of wells. In embodiments, nutrient fluid flows into the wells from the microfluidic channels on the inflow side, and waste fluid flows out of the wells from the microfluidic channels on the outflow side. In embodiments, the microfluidic channels are in fluid communication with at least one manifold, allowing fluids to be introduced into or removed from the system. The manifold may be in fluid communication with at least one external reservoir. There may be an access port in the manifold allowing access to the fluid without interrupting its flow. In embodiments, the manifold may interface with a fluid delivery system in fluid communication with the plurality of microfluidic channels that provides inflow fluid thereto.

In embodiments, the wells are constructed as through-holes through the block, being closed off inferiorly by the attachment of the block to the baseplate. Alternatively, the wells may be formed as partial bores, or cavities, having their base within the block or block-baseplate combination. The wells may be constructed to be substantially vertical, or they may be constructed at any appropriate angle for the containment of the embryos. For example, an angled construction of the wells relative to the horizontal surface of the block may prove more advantageous for introducing and/or removing embryos from the wells. In embodiments, the diameter of the wells is substantially constant along their length. In other embodiments, there may be variations in the diameter of the wells, for example, dilatations and/or constrictions, to allow the embryos to reside securely in the wells without displacement while being nourished with the nutrient fluid flowing by.

The microfluidic system may include channels of substantially even caliber, or may include channels with varying calibers, including constrictions and dilatations, as required by the fluid flow dynamics. For example, a dilatation in a channel may create a stagnant area of fluid containment, as a “lake” or reservoir of nutrient fluid that may advantageously support an embryo. As another example, a constriction in a channel may create a localized area of higher velocity fluid flow. In embodiments, constrictions may be positioned where an outlet microfluidic channel joins the well, to prevent the supported cell residing in the well from being washed into the outlet channel. In other embodiments, the microfluidic channels are dimensionally adapted to prevent the supported cell from entering them. In embodiments, the inflow microfluidic channel increases in diameter as it approaches and enters the cell well, forming a transition zone from the microfluidic channel to the cell well. In embodiments, there is a corresponding transition zone between the cell well and the outflow channel.

The principles of fluid flow dynamics may be used to improve the inflow of nutrient solutions or the outflow of waste materials, as would be appreciated by those having ordinary skill in the art. In embodiments, the flow of fluid may be dynamically regulated by valves, constrictors and the like, so that the rate of fluid flow to the embryo-bearing well can be adjusted to optimize the physiological environment. In embodiments, the microfluidic channels may flow in a direct path to the wells. In other embodiments, the microfluidic channels may be directed to cover a longer pathway, for example in a serpentine arrangement, in order to increase resistance to fluid flow.

In embodiments, the microfluidic channels may be constructed as a set of recesses on the inferior aspect of the block, having fluid communication with one or more wells. The channels may be closed off by the attachment of the block to the baseplate. In embodiments, there is one inflow channel and one outflow channel per well, although other configurations may be envisioned. In embodiments, the inflow and outflow channels are at the same level relative to each other, for example entering and exiting at the base of a well. In other embodiments, the inflow and outflow channels may enter and leave the well at a higher point, while still being constructed at the same level relative to each other. In yet other embodiments, the inflow channel may be positioned higher on the wall of the well than the outflow channel, so that gravity may assist the drainage process, or so that the inflow to outflow fluid path may best support the embryo. In other embodiments, the inflow and outflow channel is connected by “bypass” channel that can divert a partial volume of media to reduce flow rate and/or can divert air bubbles in the circuit, which may be detrimental to the supported cell, away from the wells. In other embodiments, there can be a bubble port on the inflow side that allows any air bubbles in the circuit to exit the circuit before entering the cell well.

In another embodiment, the wells, constructed as through-holes in the block, may be mated to depressions or well extensions in the baseplate, so that the well extends through the block into the baseplate. In this embodiment, the microfluidic channels may be constructed in the block, for example on its inferior aspect, or in the baseplate, for example on its superior aspect, or both. The junction of the block and the baseplate may seal the microfluidic channels constructed in either the block or the baseplate. In embodiments, the inferior aspect of the wells interfaces with a corresponding series of recesses in the base plate.

In other embodiments, the wells may be constructed as partial bores through the block that do not penetrate the inferior aspect of the block. In such embodiments, a baseplate would be optional, as the baseplate would not be needed to seal the wells inferiorly. According to such technology, the microfluidic channels would be constructed within the block as it is formed, or would be constructed to penetrate the block after it is formed, in order to achieve fluid communication with the wells. In yet other embodiments, the wells and microfluidic channels can be constructed by other micromachining processes known in the art, such as injection molding, layering, etching or laser etching, lithography, and the like.

In embodiments, the block may be made from materials like polycarbonate or polystyrene that would be compatible with the embryos residing in the wells. The baseplate may be made from similar materials, or from glass or metallics. However, other materials may be used for the block and/or baseplate. Materials selected for the block and the baseplate may be chosen in light of the type of microscopy that would be used to inspect the embryos. For example, if the materials are not optically clear, an observer may use an upright microscope to inspect the embryos, positioning the microscope to look down into the wells from the superior aspect of the block. The materials selected for the block and the baseplate should advantageously be sterilizable by conventional methods, e.g., heat or gas sterilization, and should be non-toxic to the embryo. In embodiments, the system may be reusable. In other embodiments, the system may be disposable.

In embodiments, the celltray used in the microfluidic cell support system may be formed from a block and a baseplate that are bonded together after being patterned with features such as microfluidic channels. Substrate materials for the block and/or the baseplate may include fused silica, soda-lime glass, silicon, germanium, sapphire, polystyrene, polycarbonate, and the like. Substrate materials may be selected depending on particular physical properties, including the desired optical transmission properties or electromagnetic radiation at a particular wavelength. Substrate materials may also be chosen based upon desirable chemical or fluidic control properties such as hydrophobicity and/or hydrophilicity and/or gas permeability.

The block may be attached to the baseplate using any number of techniques familiar to those of ordinary skill in the art. For example, a RTV (room temperature vulcanization) adhesive may be used. Other adhesives or bonding techniques may be selected that ensure the water-tightness of the bond between the block and the baseplate, while not leaching into the embryo support wells or the microfluidic channels.

In embodiments, the microfluidic cell support system may include a housing having an interior volume therein, within which the block bearing the cell wells may be confined. Such a housing may offer protection to the embryos in the cell wells and may provide a controlled environment for them. In embodiments, the housing may include a cover that opens and closes, for example by a hinge mechanism, a sliding mechanism, another rotational mechanism, or the like. The cover may permit access to the embryos in the wells as desired by the technician, but may then be sealed to protect the interior environment. In embodiments, the cover of the housing may be optically transparent, so that microscopy may be used to inspect the embryos without removing the celltray from the housing.

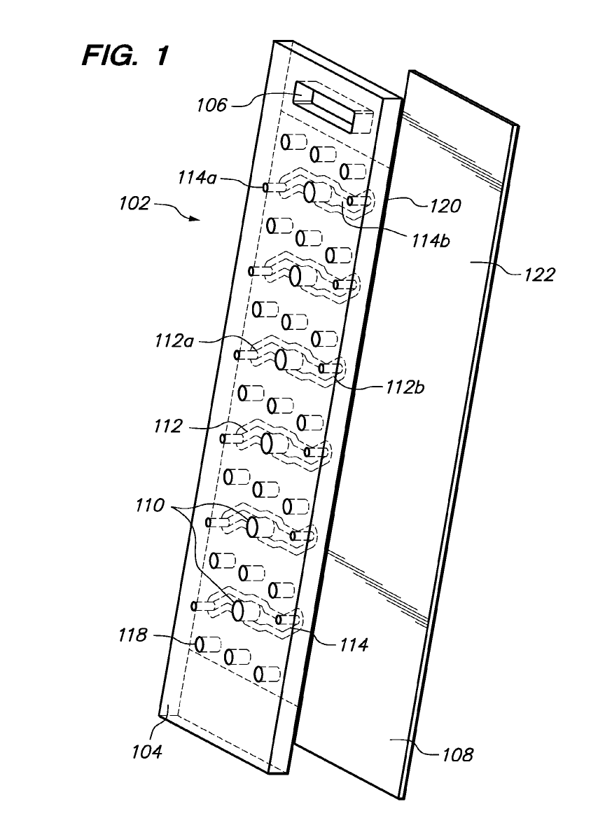

FIG. 1 depicts an embodiment of a microfluidic celltray 102 used in an microfluidic cell support system. In embodiments, the microfluidic celltray 102 includes a block 104 and a baseplate 108. There is also a notch 106 that can facilitate handling of the microfluidic celltray 102, or that can allow it to be stabilized. In the depicted embodiment, there are a plurality of wells 110 into each of which at least one living cell, for example an embryo (not shown), may be placed. In embodiments, wells 110 may be of any suitable size or shape, for example, cylindrical or square or polygonal.

Desirably, the living cell is not attached and does not adhere to the side walls of a well 110. Rather, the living cell rests freely at the bottom of the fluid contained within the well 110, or may float in the fluid without contacting the bottom or sides of the well. Such an environment can simulate the natural physiology that the living cell would encounter in vivo, for example an embryo residing in the Fallopian tubes after fertilization. In the depicted embodiment, microfluidic channels 112 are in fluid communication with the wells 110 centrally. The microfluidic channels 112 include an inflow channel 112a and an outflow channel 112b. Each microfluidic channel 112 is in fluid communication peripherally with a perfusion port 114. As shown in this figure, the perfusion port 114 system includes an inflow port 114a and an outflow port 114b.

The wells 110 of the microfluidic celltray 102 are interconnected by a microfluidics network with channels 112 that are hundreds of microns deep and contain microliter volumes of fluid. In embodiments, a microfluidics channel 112 may be 300 microns deep or more. In embodiments, the width may range from 800 to 2000 microns. Channel depth may be designed appropriately for the volume of fluid delivered to an embryo and also for containment of the embryo in the well. Because the embryos residing in the wells 110 are not adherent therein, the microfluidic channels 112 are advantageously sized smaller than the embryo, so the embryo is not flushed out of the well 110 with fluid flow. In embodiments, a support matrix block 104 bearing microfluidic channels 112 may have a thickness of approximately 3 millimeters; the relative depth of the microfluidic channels 112 to the support matrix thickness is selected to preserve the structural integrity of the device.

In embodiments, “bypass” channels (not shown) may be connected to the inflow and outflow channels to divert volumes of media (reduce flow rate) and air bubbles. The “bypass” channels may or may not be structured with similar dimensions as the inflow and outflow channels. Another method to divert air bubbles away from the well is a through-hole on the inflow channel before to the well that acts as a bubble trap or bubble port. In embodiments, the partial pressure differential between the inflow channel and the “bubble trap” can advantageously “trap” or sequester an air bubble before it enters the well where the living cell is contained.

In the depicted embodiment, a series of injection ports 118 allow the injection of a bonding agent or an adhesive such as a RTV (room temperature vulcanized) adhesive, permitting bonding between the block 104 and the baseplate 108. For example, the injected RTV can extravasate from the injection ports and occupy the space (not shown) between the block 104 and the baseplate 108. A barrier 120, for example a gasket or a raised edge of the microfluidic channels 112, can maintain a minute space (not shown) between the block 104 and the baseplate 108. Into this space, an adhesive may be injected to couple the block 104 to the baseplate 108. The barriers 120 prevent the bonding agent or adhesive from entering the microfluidic channels 112.

In use, the microfluidic cell support system with the microfluidic celltray 102 may become an adjunct to in vitro fertilization. A physician may obtain ova from an egg donor and fertilize the ova using techniques familiar to those of ordinary skill in the art. The fertilized ova may then be inserted into the wells 110 of the celltray 102, said wells 110 being filled with a nutrient medium. The inflow port 114a and the outflow port 114b may be attached to an inflow manifold through which the nutrient medium enters the system and the spent medium exits the system. The manifold may be perfused by a fluid delivery system with an optional set of reservoirs, or with ports through which additional nutrients may be added. On the outflow side, there may be a fluid evacuation system that facilitates the removal of the spent medium from the system. The fluid evacuation system may be in fluid communication with an outflow manifold that is in direct fluid communication with the outflow ports 114b. On the outflow side, there may be one or more ports that permit sampling of the spent medium. With further reference to FIG. 1, after the fertilized ovum or embryo is placed in the well 110, it remains suspended in the medium in a state analogous to what it would experience during transit down the Fallopian tube. In embodiments, the embryo may remain in the well 110 for a period of time similar to its residence in the Fallopian tube, for example 4 to 5 days. The embryo may then be removed for more permanent storage, e.g., cryopreservation. While the use of the microfluidic cell support system is illustrated by reference to techniques for IVF, it is understood that analogous techniques using the microfluidic cell support system as disclosed herein can be used to support or to culture other living cells as well.

In the depicted embodiment, there are six arrays of wells 110 with supporting microfluidic channels 112. Other numbers of arrays may be positioned on the block 104, as would be understood by those having ordinary skill in the art. For example, 8 or 10 arrays of wells 110 and supporting microfluidic channels 112 may be arranged on a block 104, or other numbers of arrays. Desirably, for use in IVF, the number of wells 110 would accommodate the number of embryos harvested from an egg recovery procedure for a single IVF patient. In other embodiments, however, larger numbers of well arrays may be contained within a single microfluidic celltray 102, an arrangement that may be particularly useful for research applications and other settings where single-patient embryo support is not involved, and/or where cell support for other types of cells is utilized. The systems and methods disclosed herein may permit the monitoring, for example, of large arrays of embryo cultures or other living cells. In embodiments, these systems and methods may allow researchers to inspect or otherwise investigate or study numerous embryos or other living cells, with a reduction of time, space and cost as compared to more traditional methods for embryo or other living cell culture.

In embodiments, the microfluidic celltray 102 is sized and shaped similarly to an ordinary microscope slide. This permits ready examination of the resident living cells, e.g., embryos, using routine microscopy. Embodiments of the present invention may include two-dimensional arrays of millimeter-sized wells containing, for example, embryos for IVF and the nutrient solutions to support them. Such an arrangement may enable automated processing as well as simultaneous monitoring and analyzing of a collection of living cells, e.g., embryos. These arrays may be micromachined to be housed on a slide measuring 76.2×25.4 mm, e.g., the size of a standard microscope slide. Wells are advantageously scaled to millimeter sizes, for example, having volumes between 4 and 12 microliters, although it would be understood in the art that smaller wells could be provided in accordance with available methods for microfabrication if such wells could be serviced by ancillary devices such as micropipettes and sealing rings that were similarly miniaturized.

Arrays configured for use with conventional microscopy may be designed with additional features to facilitate their handling. For example, a celltray bearing an array may be configured with corners that are rounded or with indentations on the edges to allow easy pick-up or manual manipulation by an operator. Vertical tabs for the celltray can be provided via the machining, molding, or by bonding that facilitate the use of automated grippers in slide handling robots. The celltray may also be sized to rest on pedestals formed, for example, within a Petri-dish-like holder, permitting ready manipulation. In embodiments, the celltray may be encased within a housing that maintains a protective environment around it. The microfluidic cell support system disclosed herein is compatible with such celltray modifications and additional features. While these systems and methods will be described by reference to an array sized to fit on a standard microscope slide, it is understood that other sizes and shapes of the array's housing may be produced to fit specific industry demands. While a small physical footprint is advantageous for certain purposes, it would be understood in the art that the housing may be formed in any size or shape to fit a particular piece of apparatus, or to provide a sufficiently large matrix for analytic purposes.

Wells for the microfluidic cell support system are open on their top surface, so that the wells may be readily seeded with living cells like embryos, for example under a laboratory hood. In embodiments, the openings of the cell wells may be machined in advantageous shapes, e.g., chamfered or funneled, to facilitate the easy placement of cells therein. Following cell placement, the celltray may be covered by a manifold assembly, a housing, or by any appropriately sized and shaped cover so that the embryos in the celltray may be incubated. In embodiments, multiple celltrays may be seeded at one time in a laboratory hood or similar facility without a pump or a manifold, due to the open construction of the wells. It would be appreciated by one of ordinary skill in the art, however, that celltray seeding may take place with the tray placed on the microscope stage or in any convenient place. The celltray may be pre-calibrated before seeding, to permit passive auto-focus, well-to-well navigation, microscopic scanning, and the like. The open configuration of the wells in the celltray also allows for access to the wells before or after seeding, for a variety of purposes as would be appreciated by those of ordinary skill in the art.

Wells may be placed in discrete arrangements within regions on the celltray. Each region may be configured with different well sizes or shapes, or different microfluidic properties. Such regional differences permit discrete, uniform sample populations to be created, so that multiple parallel isolated embryo cultures may be conducted. In embodiments, six regions of single wells may be arrayed on a celltray. In other embodiments, increased regions of single wells may be provided, for example, 8 or 10 regions. In other embodiments, regions may be arranged with any number of wells, for example with 2 wells, 4 wells, or a multiple thereof. It will be apparent to those of ordinary skill in the art that other arrangements of wells and regions may be provided, consistent with the needs of particular cell support protocols.

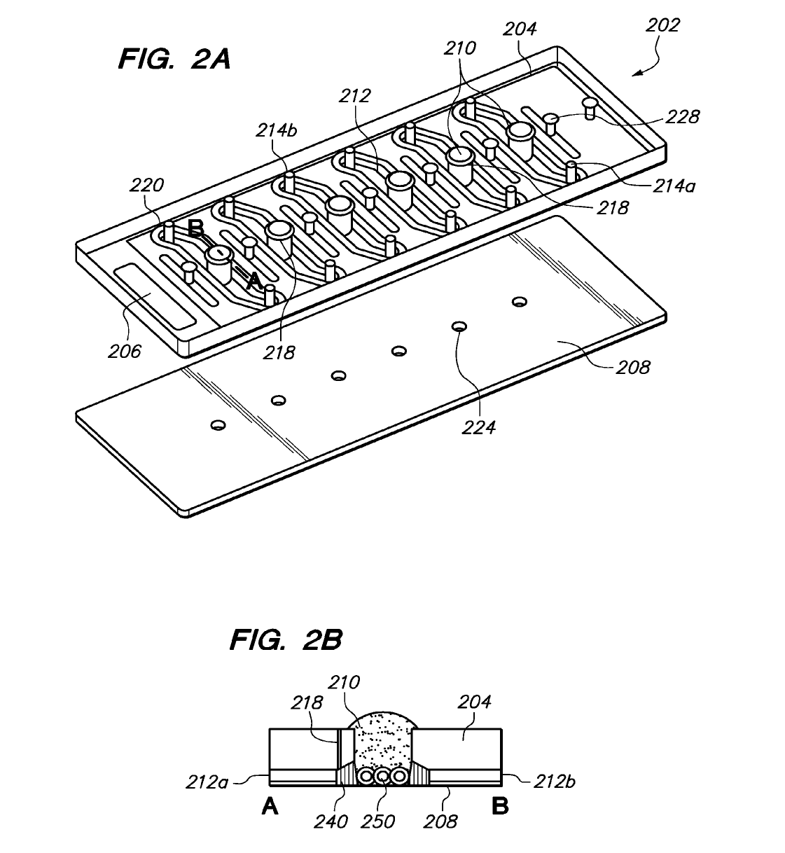

FIG. 2A depicts an embodiment of a microfluidic celltray 202 as used in a cell support system. In FIG. 2A, a microfluidic celltray 202 includes a block 204 and a baseplate 208. The block 204 is optionally equipped with a notch 206 to facilitate handling. The block 204 bears a plurality of cell wells 210 configured as through-holes bored through the block 204 and interfacing with a corresponding series of recesses 224 bored in the baseplate 208. As shown in the depicted embodiment, the outer edge of the wells 210 can be flared or chamfered to facilitate introduction of the cells to be supported within the well system. Each cell well 210 is in fluid communication with a set of microfluidic channels 212 bringing fluid into the cell well 210 on the afferent side and removing fluid from the cell well 210 in the efferent side. The microfluidic channels 212 are in fluid communication with an inflow port 214a on the afferent side, and an outflow port 214b on the efferent side. On the afferent side of the microfluidic channels 212, there is a bubble port 218 that can trap air bubbles that may be within the fluid circuit. This mechanism can prevent air bubbles from being transported into the cell well 210 where they could contact the cells residing therein (not shown). In the depicted embodiment, there are multiple injection ports 228 for injecting an adhesive agent to bond the block 204 to the baseplate 208. The adhesive can extravasate from the injection ports 228 into a minute open space between the block 204 and the baseplate 208, while being prevented from entering the microfluidic system 212 by a series of barriers 220 that surround each microfluidic system 212 to wall it off.

FIG. 2B shows a diagram of a portion of the celltray 202 in FIG. 2A, taken to show a cross-section of FIG. 2A from point A to point B. FIG. 2B shows a block 204 attached to a baseplate 208, with a cell well 210 penetrating the block 204 to communicate with a recess 224 in the baseplate 208. The cell well 210 is shown to have a flared external aspect, making it easier to deposit cells 250 therein. As shown in this diagram, the cells 250 have sunken to the lower portion of the cell well 210 where it communicates with the recess 224. The cells 250 may occupy a variety of positions in the cell well 210, depending in part on their response to the fluid flow therethrough. A system of microfluidic channels communicate fluidically with the cell well 210, including an afferent microfluidic channel 212a and an efferent microfluidic channel 212b. As the microfluidic channels enter and exit the cell well 210, there is a transition zone 240 where the microfluidic channel widens as it approaches the cell well 210. Proximal to the afferent transition zone 240 is a bubble trap 218. Advantageously, the orifice of the bubble trap 218 on the superior aspect of the celltray 202 can be confluent with the orifice of the cell well 210.

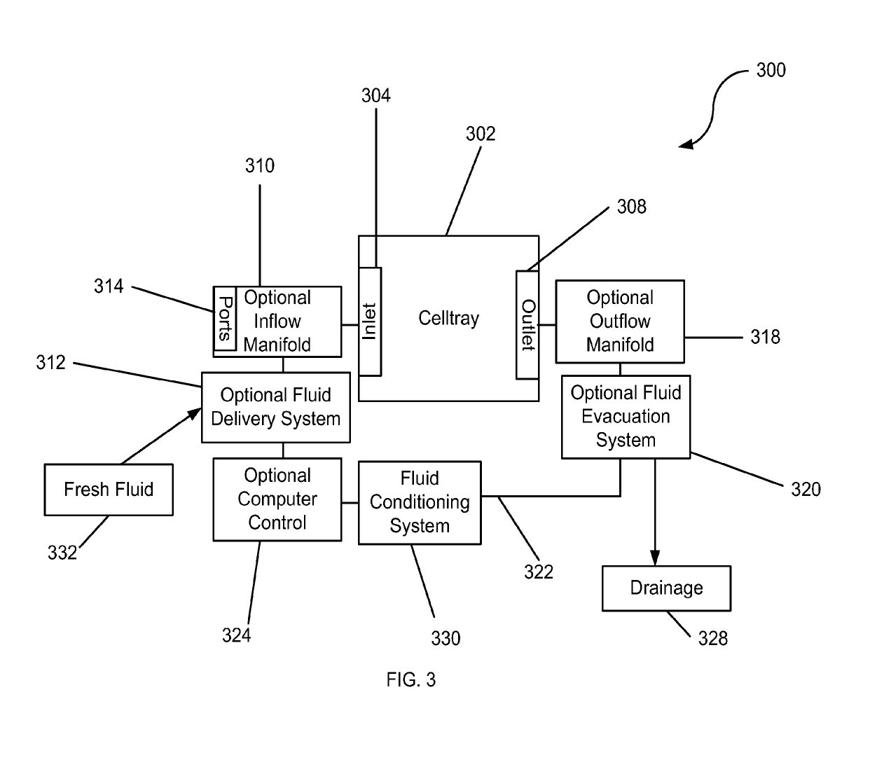

FIG. 3 provides a block diagram of a system for fluid flow management that can be used with a microfluidic celltray. The system 300 shown in FIG. 3 is intended to provide fluid to and remove fluid from a celltray 302. The celltray 302 can have an inlet 304 and an outlet 308 in fluid communication with the celltray 302 microfluidic system (not shown) and in fluid communication with a fluid delivery system 312 and a fluid evacuation system 320. In embodiments, the passage of fluid throughout the system 300 is controlled by a computer 324.

As depicted in FIG. 3, the fluid can pass from the fluid delivery system 312 into the inflow manifold 310, thereby entering the celltray inlet 304. The fluid passes through the celltray 302, exiting at the outlet 308 and passing into the outlet manifold 314. At the inflow manifold 310, there can be one or more ports 316 allowing the introduction of reagents into the inflow fluid path. At the outflow manifold 314, there can be one or more ports 318 allowing the removal of fluid from the outflow fluid path, e.g., for testing, assays or other sampling purposes. Fluid can pass through the outflow manifold 314 into the fluid evacuation system 320, where it can be drained off 328, or where it can be recycled 322 for further use in the system. Fluid to be recycled 322 can be passed through a fluid conditioning system 330, where it can be prepared for subsequent use as inflow fluid. As shown in the diagram if FIG. 3, the fluid conditioning system 330 can be in line with the fluid flow circuit. The fluid conditioning system 330 can also take the fluid offline for conditioning, returning it via a separate pathway into the fluid flow circuit. The recycled fluid can be combined with fluid entering from a fresh fluid source 332. In other embodiments, all fluid is provided through the fresh fluid source 322. In yet other embodiments, all fluid is recycled after the initial infusion of fluid from the fresh fluid source 332.

In embodiments, the microfluidic cell support system may be placed within the manifold assembly. So positioned, the wells of the system are accessible from the outside, so that the system is mechanically open. So positioned, the microfluidics of the celltray are in fluid communication with an external fluid delivery system providing fluid inflow and outflow, with the path for fluid circulation being a closed one.

The manifold provides an interface between the microfluidics of the celltray and an external fluid delivery system that may provide for fluid infusion and fluid withdrawal from the celltray microfluidics. The celltray may be configured with access ports that interconnect to the manifold, for example via O-ring connections. The manifold may be equipped with quick release fasteners to control preload on the O-rings to achieve a reliable, fluid-tight seal. Guide pins and the like may be provided on the manifold to align it properly with the celltray. In embodiments, the manifold may be fabricated from biocompatible materials, for example, polycarbonates, polymethylpentene, and amorphous thermoplastic polyetherimide (e.g., Ultem®), and the like. In embodiments, the manifold may be autoclavable. In embodiments, the manifold may be opaque to minimize reflections and the like.

In an embodiment, the manifold may be shaped as a housing that includes a hinged cover glass, coated for example with indium tin oxide, to control access to the wells, to control temperature, to minimize contamination, and the like. So configured, the manifold provides a housing for the celltray within which a controlled environment may be maintained. Temperature control may be facilitated by an integrated temperature feedback mechanism. A liquid (water) reservoir attached to the manifold may permit humidification of input gas. An air port on the manifold may permit humidified, regulated gas flow into and out of the controlled environment. The manifold may contain infusion ports directed, for example to the various regions of wells on the celltray, so that different fluids or reagents may be added to each region. Such infusion ports may be closed during normal operations, for example with a cover, a diaphragm or a one-way check valve. Consistent with the regional design of the celltray, the manifold may be organized into regions as well. Regions within the manifold may be multiplexed, for example, to minimize tubing connections. Regions may also be isolated from each other to permit isolated experiments from region to region. Other features may be incorporated into the manifold to accommodate specific experimental needs, as would be apparent to persons having ordinary skill in the art.

Advantageously, the manifold may be constructed so that it interfaces with commercially available fluid delivery system components, such as the tubing that are gas impermeable, semi-permeable, or permeable and fittings used for high-pressure liquid chromatography and the like. In embodiments, the fluid delivery system may include a pump or a series of pumps to control fluid inflow and outflow. Pumps may be compactly made, so that they can fit conveniently on or under a laboratory bench. Pumps may, in embodiments, use commercially available syringe pumps for precision and reliability. In embodiments, miniaturized stand-alone pumps may be used that would be suitable for use in an incubator or under a laboratory hood. The pumps may be controlled from a computer that is controlled from a user interface via, for example, a USB or other connection, in accordance with a fluid delivery program that regulates the fluid delivery system. The computer connections permit a plurality of fluid delivery systems to be controlled for a plurality of microfluidic cell support systems. In embodiments, a fluid delivery program may include a series of preset routines for fluid delivery, including priming, feeding, infusing and purging the microfluidic channels of the celltray. In embodiments, continuous and pulsed pumping modes may be available, including configurable pulse delays. A range of flow rates may be controllable, with pump rates ranging from 5 μl/hr-50 μl/hr continuous, for example, or in a pulsed mode. In embodiments, a pulsed flow may be calibrated so that fluid is pumped into and withdrawn from the embryo wells on a timed basis. For example, fluid inflow can be pulsed in intervals having a duration of seconds and a frequency of a certain number of cycles per minute; in embodiments, fluid inflow intervals may be longer or shorter as needed, and frequencies may be set in accordance with the physiological needs of the embryo. In one embodiment, 0.1 microliters of fluid could be exchanged per pulse.

Offsets may be determined to compensate for environmental losses of fluid, via evaporation for example. Flow rates and volumes may be configured via the user interface. The fluid delivery system may further include infusion ports to allow reagents to be added to the inflow circuit, so that, for example, a set of parallel isolated experiments could be run using a single pump set. Infusion ports may be sealed from the rest of the fluid system with membranes or other sealants, or with static or dynamic valve systems, or by other mechanisms as would be understood by those of skill in the art. In embodiments, the celltray of the microfluidic cell support system may be machined to incorporate a resistance network to control the flow of fluid evenly across all the regions of the celltray. Other features may be incorporated into the fluid delivery system, for example t-valves on the outflow ports allowing waste fluid to be extracted from individual regions on the celltray.

Additional sensors may be used with the system. These can include electrical sensors embedded within the chip such as thermistors or electrodes permanently bonded between the top and bottom layers of the chip with wires routing out that can be connected to a measurement device such as a multimeter, oscilloscope, or feedback controller. Other types of sensors can include includes mechanical sensors such a liquid crystal mylar sheets or lacquer crayons where temperature changes result in a visible color change. Sensors can also be part of the fluidic path external to the chip such as an electrode inline with the outlet flow. Other sensors include relative humidity sensors mounted to the manifold that measure the environment in the enclosed incubation area (e.g., in the well access chamber described in more detail below), and external optical probes which can use spectral information to characterize the pH. Other sensors can be attached to or embedded in the microfluidic cell support system or its housing to produce data regarding parameters pertaining to the overall system or any component thereof. In addition, sensors can be integrated into the fluid inflow path and/or the fluid outflow path.

A system parameter to be measured by the sensor may include, for example, a chemistry parameter, a temperature parameter, a humidity parameter, a fluid flow parameter, or the like. After measuring the data provided by the sensor, the conditions within the microfluidic cell support system can be modified to optimize cell support. For example, temperature within the system (either fluid or ambient environment), rates of fluid flow, pressure, humidity within the ambient environment, or chemical composition of the fluid or the ambient environment can be modified.

In embodiments, the microfluidic cell support system may be adapted for use with a microscope, for example a regular microscope, an inverted microscope, or any other optical device known in the art. A control system may allow the user to operate the microscope while managing the microfluidic cell support system. In embodiments, controls could direct the well-to-well navigation of the microscope, its focus or focus offset adjustments, other camera adjustments, shutter control. The control system may also provide for cataloging and logging, configurable data indexing for import into an image analysis package, and the like.

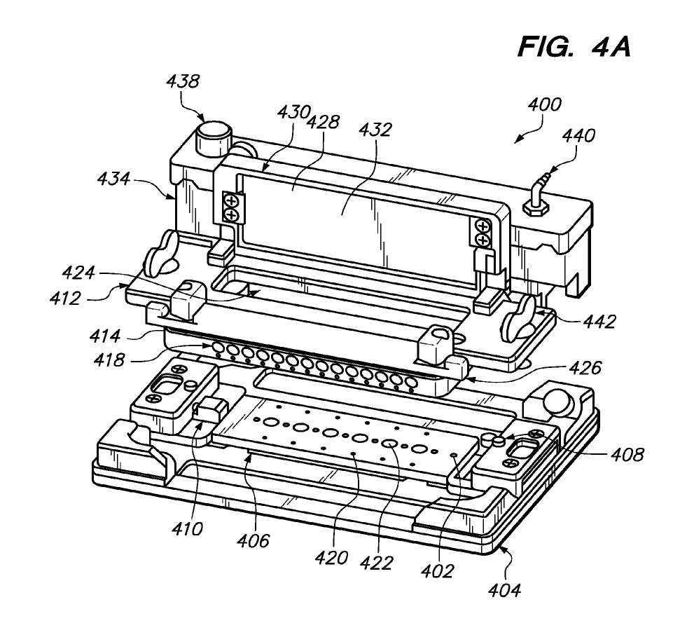

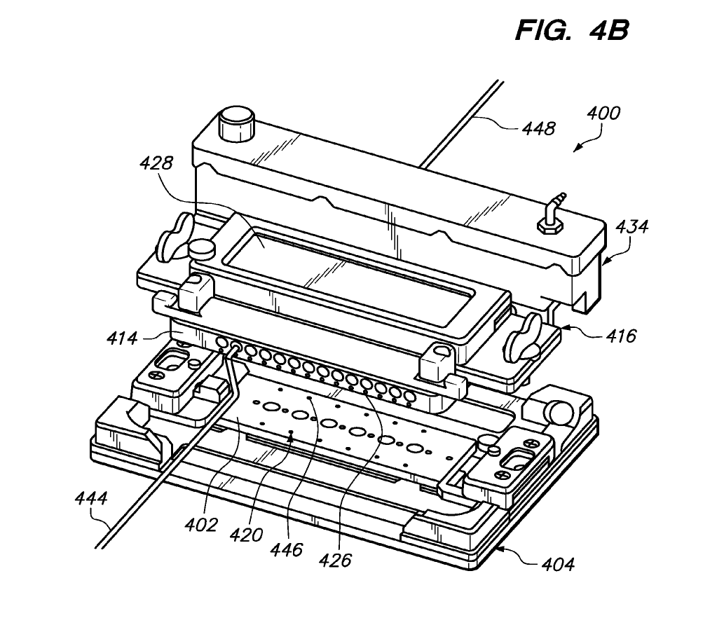

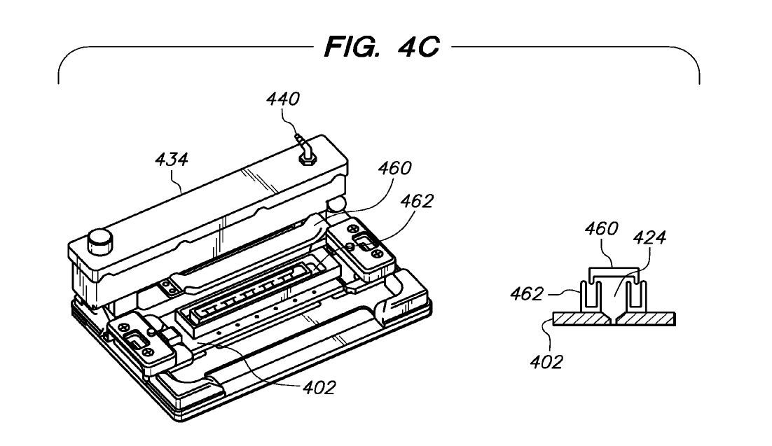

FIGS. 4A and 4B illustrate embodiments of a cell support system, comprising a celltray 402, and a housing assembly 400, which includes a base assembly 404 and a manifold assembly 412. The base assembly 404 and manifold assembly 412 can couple with each other to provide an enclosed and controlled environment for the celltray 402, with control over fluid inflow, fluid outflow, temperature and humidity. When coupled to each other, the base assembly 404 and manifold assembly 412 still permit access to the celltray 402 by means of a hinged cover glass 428 that, when open, allows one to access the open superior aspect of the cell wells 422. The housing assembly 400 thus allows the celltray 402 to remain microfluidically closed and mechanically open.

As depicted in FIG. 4A, the housing assembly 400 comprises a base assembly 404 and a manifold assembly 412. The base assembly 404 provides a supporting deck for the celltray 402. As depicted here, the supporting deck can include mounting tabs 406 upon which the celltray 402 is positioned and secured. A wedge ejector 410 is available to assist with removing the celltray 402 from its position on the base assembly 404. The base assembly 404 interfaces with the manifold assembly 412, and the two components are positioned with respect to each other by a set of alignment pins 408 that fit into receiving recesses (not shown) on the underside of the manifold assembly 412. The base assembly 404 and the manifold assembly 412 can be secured to each other by a quick release fastener 442 or comparable attachment mechanism.

The manifold assembly 412 bears the inflow manifold 414 that interfaces with the celltray inflow ports 420, and the outflow manifold 416 that interfaces with the celltray outflow ports 446. The inflow manifold supports a number of inflow ports 418 that permit delivery of fluid into the corresponding inflow ports 420 on the celltray 402. On the outflow side, the outflow manifold 416 supports a number of outflow ports (not shown) that are in fluid communication with outflow ports 446 on the celltray 402 that allow fluid to flow out of the celltray 402. The inflow manifold 414 can provide one or more access ports 426 to allow, for example, injection of reagents into the fluid flowing into the celltray. Analogously, the outflow manifold 416 can provide one or more sample ports (not shown) to allow, for example, withdrawal of fluid samples from the fluid flowing out of the celltray 402.

In embodiments, fluid media can be delivered from premixed tanks, with humidifying and gassing in the media conicals to equilibrate pH similar to standard practice in an incubator. In embodiments, the media may be heated or cooled as appropriate for a given culture protocol. The inflow manifold 414 receives fluid through one or more inflow tubings 444, and the outflow manifold 416 delivers fluid into one or more outflow tubings 448. On the inflow side, tubing 444 can enter the manifold through standard 50 ml conicals with grommets added to cap to pass tubing through and maintain seal.

In embodiments, the inflow and/or the outflow tubing can be made from a material that is impermeable to carbon dioxide or to other gases. In embodiments, the tubing can be fabricated from polymeric materials such as perfluoroalkoxy, polyaryletheretherketone, ethylene chlorotrifluoroethlyene, ethylene tetrafluoroethylene, multi-layered polyvinyl chloride, or from fused silica. In embodiments, the tubing is fabricated from materials having a low gas permeability to sustain proper gas (CO2) concentration, and/or a low vapor permeability to preserve osmolality, concentrations, etc., and/or low light transmission to prevent protein decomposition. In embodiments, the tubing can be optically opaque, or the entire inflow system can be optically opaque, provided for example by fabricating a thin film on the glass syringes delivering the media, an opaque sleeve on media conicals and an optically opaque delivery tubing.

The manifold assembly 412 is equipped with a hinged cover glass 428, shown in the open position in FIG. 4A and in the closed position in FIG. 4B. In embodiments, the hinged cover glass can be fabricated from glass or polycarbonate. In embodiments, it can be removed partially or entirely from the manifold assembly 412 to facilitate cleaning or replacement. When open, the hinged cover glass 428 reveals a well access chamber 424, wherein the external orifices of the cell wells 422 are exposed. The cell wells 422, containing fluid, may be in contact directly with the environment within the well access chamber 424. In embodiments, the fluid may be protected on its surface from the environment of the well access chamber 424 by a protective layer, for example a fluid like an oil or a polymer.

With the cover glass 428 closed, as shown in FIG. 4B, the well access chamber 424 environment can be controlled. Thus, the fluid in the cell wells 422 can contact an ambient environment having specialized properties that encourage cell growth. In embodiments, the environment within the well access chamber 424 can be controlled. In other embodiments, the environment within the well access chamber 424 can be the same as the external environment. When the hinged cover glass 428 is open to the external environment, the well access chamber 424 environment may equilibrate with the external environment and may lose some of its advantageous characteristics. Any specialized ambient environment within the well access chamber 424, whether maintained by optimization of gaseous environment, humidity, temperature, pressure, or the like, may be susceptible to change or dissipation if the hinged cover glass 482 is left opened for a sufficient period of time.

The housing assembly 400 can include features to control other aspects of the cell's environment. To control heating, for example, a heater assembly 430 can be included within the manifold assembly 423, for example in proximity to the cover glass 428. In an embodiment, the heating element 432 for the heater assembly can be formed in proximity to or in conjunction with the cover glass 428. In other embodiments, the heater assembly 430 can be placed wherever convenient, for example, as part of the base assembly or in proximity to the fluid-bearing portion of the cell wells. In embodiments, the heater assembly 430 can be positioned adjacent to the cell wells 422, for example, as a heating element 432 that is set into the celltray itself or that is positioned vertically to parallel the cell well 422. To control the humidity in the cell wells 422, and/ or to control evaporation therefrom, a humidifier assembly 434 can be integrated with the manifold assembly 412, for example providing a humidified environment within the well access chamber 424. The humidifier assembly 434 can provide conditioned (warmed and humidified) gas to the well access chamber 424. In embodiments, the humidifier assembly 434 can include a self-regulating positive temperature coefficient (PTC) heating element.

In embodiments, evaporative losses can be minimized by supplying heated, humidified air into the well access chamber 424. In embodiments, the well access chamber 424 can be incompletely sealed, allowing gases from the well access chamber 424 to pass into the outside environment, and ensuring that outside air does not enter into the well access chamber 424. Small amounts of evaporation may be compensated for by using a slight offset between the volume dispensed and withdrawn from the celltray 402.

As shown in FIG. 4C, evaporation may be further reduced through the use of an evaporation reservoir 462 and an evaporation lid 460 defining the well access chamber 424. In embodiments, the evaporation lid 460 can be mechanically fastened to the within the incubation region. The evaporation lid is mechanically fastened to the cover glass 428 or the heater assembly 430. The evaporation reservoir 462 and lid 460 can function in an incubator like a Petri dish, allowing slow gas exchange to the celltray 402 but maintaining a high humidity in the air directly above the open wells.

A gas inflow port (not shown) can direct humidified air or other appropriate gas media into the well access chamber 424, where the gaseous environment can be maintained at the desired humidity, pressure and gas composition. Gas mixtures can enter the humidifier assembly 434 through a controlled gas inlet 440, and humidifying water can be introduced through the humidifier refill ports 438.

While the invention has been described in connection with certain embodiments, other embodiments would be understood by one of ordinary skill in the art and are encompassed herein. Thus, one of ordinary skill in the art will readily recognize that there could be variations to the embodiments, and any variations would be within the spirit and scope of the present application. Accordingly, many modifications may be made by one of ordinary skill in the art without departing from the spirit and scope of the method and system disclosed herein.

We claim:

1. A microfluidic cell support system, comprising
a base assembly,
a manifold assembly, and
a microfluidic celltray including a microcirculatory path in fluid communication with the manifold assembly,
wherein the microfluidic celltray is microfluidically closed and mechanically open.

2. The system of claim 1, wherein the microfluidic celltray includes at least one cell well containing fluid for nourishing at least one living cell, the fluid having a surface in contact with a gaseous environment.

3. The system of claim 2, wherein the fluid comprises a nutrient fluid for culturing the at least one living cell.

4. The system of claim 2, wherein the at least one living cell is an embryo.

5. The system of claim 2, wherein the gaseous environment is an ambient environment.

6. The system of claim 5, wherein the manifold assembly comprises a hinged cover glass which, when closed, encloses the ambient environment.

7. The system of claim 5, wherein the ambient environment is a specialized environment with a controlled variable selected from the group consisting of gas composition, humidity, temperature and pressure.

8. The system of claim 1, wherein the microcirculatory path comprises a microfluidic inflow channel and a microfluidic outflow channel with an active fluid flow therebetween.

9. The system of claim 1, further comprising a sensor circuit to produce data regarding at least one system parameter.

10. The system of claim 9, wherein the at least one system parameter is selected from the group consisting of a chemistry parameter, a temperature parameter, a humidity parameter, and a fluid flow parameter.

11. A method for supporting a living cell, comprising:
providing the microfluidic cell support system including a base assembly, a manifold assembly, and a microfluidic celltray including a microcirculatory path in fluid communication with the manifold assembly, the microfluidic celltray being microfluidically closed and mechanically open, wherein the microfluidic celltray further includes a cell well;
providing a nutrient fluid selected for supporting at least one living cell; and
perfusing the cell well with the nutrient fluid, and introducing the at least one living cell into the cell well containing the nutrient fluid, whereby the step of perfusing includes establishing an active fluid flow from a microfluidic inflow channel to a microfluidic outflow channel across the cell well.

12. The method of claim 11, wherein the at least one living cell is an embryo.

13. The method of claim 11, wherein the cell well is microfluidically closed and mechanically open.

14. The method of claim 11, wherein the nutrient fluid in the cell well is in contact with an ambient environment.

15. The method of claim 14, further including the step of regulating at least one system parameter for the microfluidic cell support system to optimize cell support.
